(19) United States
(12) Plant Patent Application Publication
(10) Pub. No.: US 2008/0201811 P1
(43) Pub. Date: Aug. 21, 2008

(54) Fallopia plant named 'Igniscum'

(75) Inventor: Maria Rogmans, Kalkar (DE)

Correspondence Address: LERNER GREENBERG STEMER LLP, P O BOX 2480, HOLLYWOOD, FL, 33022-2480, US

(73) Assignee: Conpower Energie GmbH & Co. KG

(21) Appl. No.: 12/069,926

(22) Filed: Feb. 14, 2008

(30) Foreign Application Priority Data
Feb. 15, 2007 (EM) CPVO 2007/0149

(51) Int. Cl. A01H 5/00

(52) U.S. Cl. PLT/263.1

(57) ABSTRACT

A new and distinct cultivar of Fallopia plant named ‘Igniscum,’ characterized by foliage which is elliptical, with a twisted top, a straight line at the lower margin, a leaf color of moss green with a velvety upper surface texture at leaf maturity. The lower surface is coriaceous, the leaves are very firm and with little moisture. The margins of the leaves are smooth, the lateral nerves connect at the side of the leaf in a roof-tile structure. The leaf stalks are burgundy even at maturity. The blossoms are creamy white.

### CROSS-REFERENCE TO RELATED APPLICATION

This application claims the priority, under 35 U.S.C. § 119, of European Union Plant Variety Patent Application CPVO 2007/0149, filed Feb. 15, 2007; the prior application is herewith incorporated by reference in its entirety.

### BOTANICAL CLASSIFICATION

Polygonaceae, class of Posopside, Caryophyllidae

### CULTIVAR DESIGNATION

‘Igniscum’

### BACKGROUND OF THE INVENTION

1. Field of the Invention

The present invention, ‘Igniscum,’ relates to a new and distinct cultivar of Japanese Knotweed Fallopia or Mexican Bamboo/Round Knotweed, botanically known as Fallopia Sachalinese and hereinafter referred to as ‘Igniscum.’

The ‘Igniscum’ plant is native to Sakhalin Island in the South Kuril Islands and over the years became naturalized in Europe. The species ‘Igniscum’ is a variety that was discovered in the wild, is particularly fast-growing, has no tillers, is hardy and is a producer of a great deal of biomass. The plant ‘Igniscum’ was discovered in 1987 at the location of the breeder, Kräuterey Wilhelm, Spierheide 54, D-47546 Kalkar, Germany, as a spontaneous mutation of the original Fallopia Sachalinense with differences in the exterior and in the roots and the seeds. Isolation/separation from other plants and development of the species was then carried out at the breeder's location over 20 years, during which the entire procedure was kept secret. Only the method of division of parts of the roots was used for generation, because the new species is not intended to produce seeds.

The result of this development was that this new species, designated ‘Igniscum’ by the inventor, had lost its detrimental characteristic of being invasive. In other words, the new species ‘Igniscum’ is not invasive due to the fact that it has no running roots, and no seeds or only very few seeds. This means that ‘Igniscum’ can now be cultured normally on a plantation. This is a very important, new and surprising feature which makes ‘Igniscum’ very different from its genetic parent ‘Fallopia Sachalinense.’

‘Igniscum’ is maintained by using asexual propagation methods, e.g. cuttings, meristem propagation and cell cultures, at Kräuterey Wilhelm, at the breeder's location in Kalkar, Germany mentioned above.

Due to this special unusual feature of the new ‘Igniscum’ plant variety, of it only being possible to be reproduced through asexual propagation, which is completely different from the origin Taxon species Fallopia Sachalinense, the new ‘Igniscum’ plant variety is non-invasive.

Therefore, it is a surprising result that the requirement for asexual propagation leads to the extremely advantageous effect of non-invasiveness. This is surprising because the origin plant Sachalinense is believed to be the most invasive plant on earth, while the new variety ‘Igniscum’ is not invasive. Due to this fact, the new ‘Igniscum’ plant variety can be cultured on large, open terrain.

Furthermore, the new ‘Igniscum’ plant variety is extremely uniform and stable, which has been verified during the last 20 years of development of the plant.

With regard to reproduction, experience has shown the plant to be sterile. Genuine propagation can only be accomplished by shoots, meristem propagation or cell cultures.

Exploitation of the plant is as an industrial crop or energy crop, which is usable in biological plant protection and in health care.

Regarding duration of the culture, the plant is a perennial species. The inventor has been experimenting with the plant for 20 years and has seen that the biomass yield increases annually. Reynoutria sachalinense has been known to be a wild species to this day.

The inventor is not aware of any addresses and collections of varieties.

### BRIEF SUMMARY OF THE INVENTION

The new plants of the hybrid variety of Renoutria ‘Igniscum’ have been observed repeatedly for twenty years as having unique traits. These characteristics, in combination, distinguish ‘Igniscum’ as a new and distinct cultivar, as follows:
1. The foliage of the ‘Igniscum’ is elliptical, with a twisted top, a straight line at the lower margin, a leaf color of moss green with a velvety upper surface texture at leaf maturity. The lower surface is coriaceous, the leaves are very firm and with little moisture. The margins of the leaves are smooth, the lateral nerves connect at the side of the leaf in a roof-tile structure. The leaf stalks are burgundy even at maturity. The leaf size varies between a breadth of 13 cm and a length of 21 cm and a breadth of 17 cm and a length of 22 cm. The blossoms are creamy white.
2. The height of the ‘Igniscum’ plant is 3-4 m.
3. The ‘Igniscum’ plant is hollow with internodal segments, high fiber content and lignification in late summer/autumn.
4. The ‘Igniscum’ plant has no tillers, sends out runners easily, and its rootstock reaches down to a depth of 2 m. There are 30-100 stems per plant and approximately 20-22 nodes.
5. The blooming time, depending on the weather, may start in July, but is usually between August and November.

### BRIEF DESCRIPTION OF THE SEVERAL VIEWS OF THE DRAWING

The colored photographs described below illustrate the overall appearance of the new cultivar, showing the colors as true as it is reasonably possible to obtain in such colored reproductions. The colors in the photographs may differ from the color values mentioned in the detailed botanical description, which accurately describes the colors of the new ‘Igniscum’ plant.

FIG. 1 is a photograph showing a branch of ‘Igniscum;’

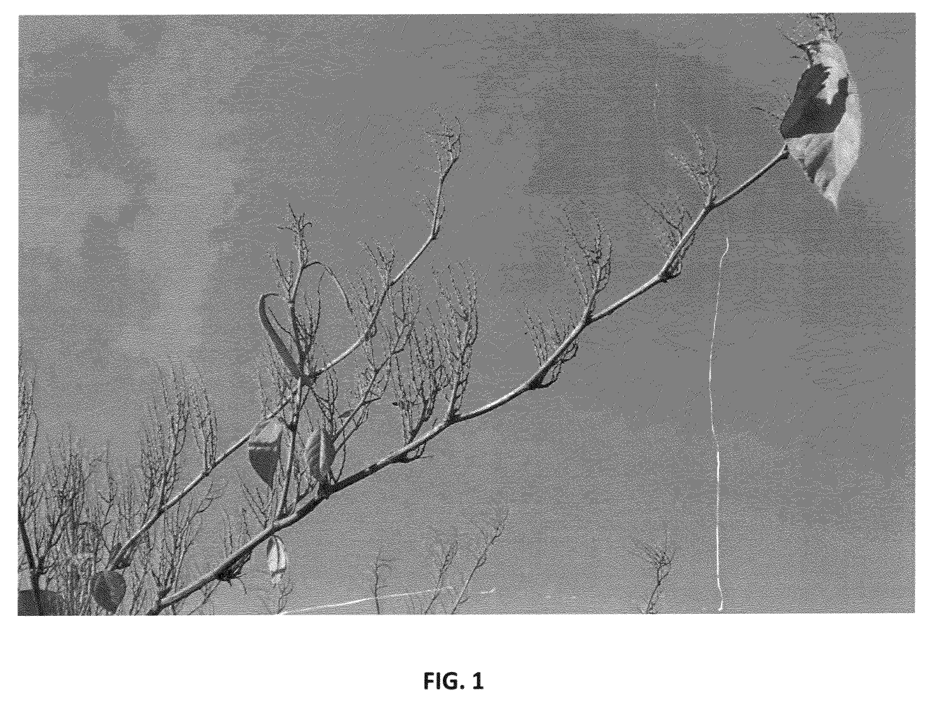

FIG. 2 is a photograph showing several ‘Igniscum’ plants;

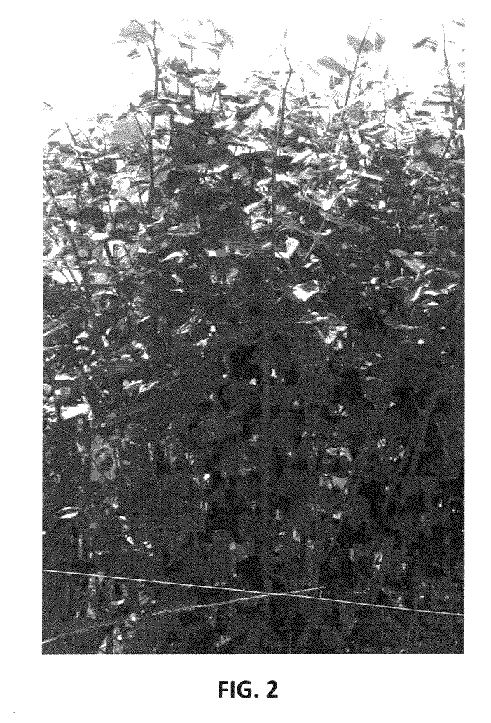

FIG. 3 is a photograph showing an enlarged view of ‘Igniscum;’

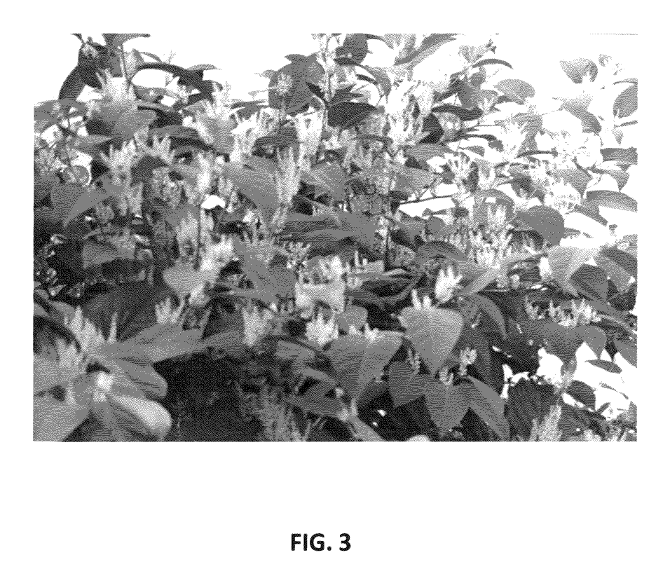

FIG. 4 is a photograph showing an enlarged view of a branch of ‘Igniscum;’

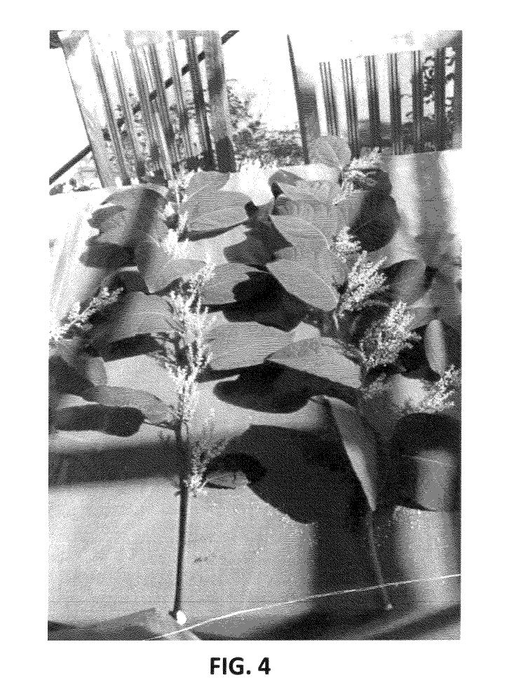

FIG. 5 is a photograph showing a view of ‘Igniscum’ similar to FIG. 3 on a reduced scale;

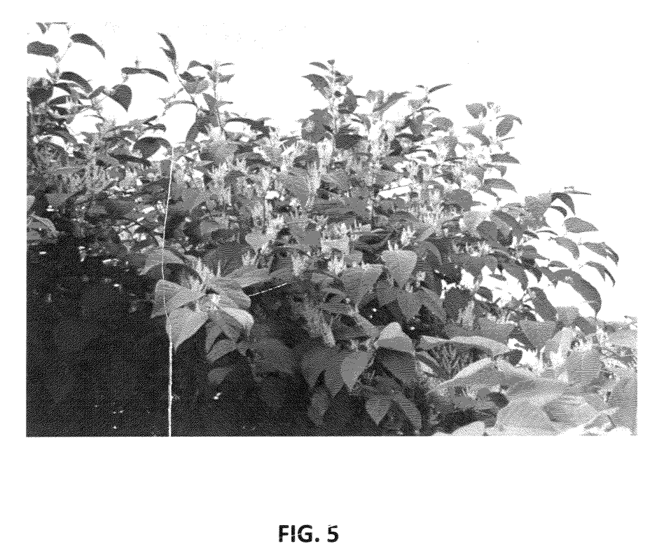

FIG. 6 is a photograph showing another view of ‘Igniscum’ similar to FIG. 3;

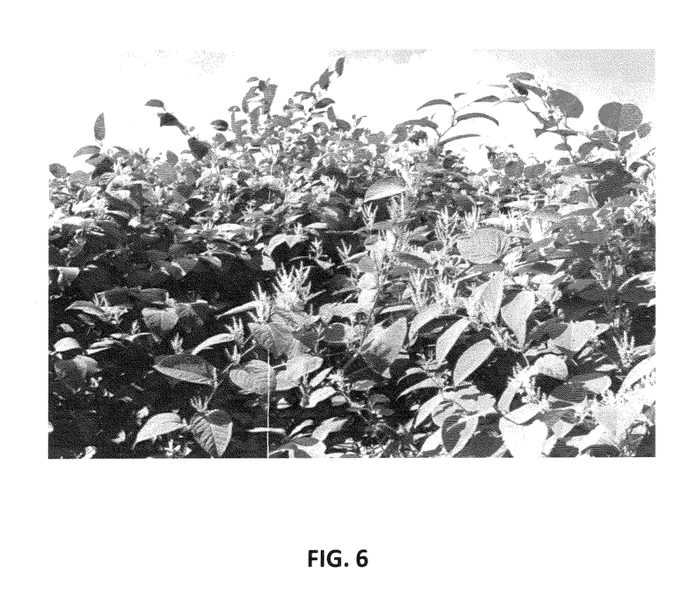

FIG. 7 is a photograph showing an enlarged view of several leaves of ‘Igniscum;’

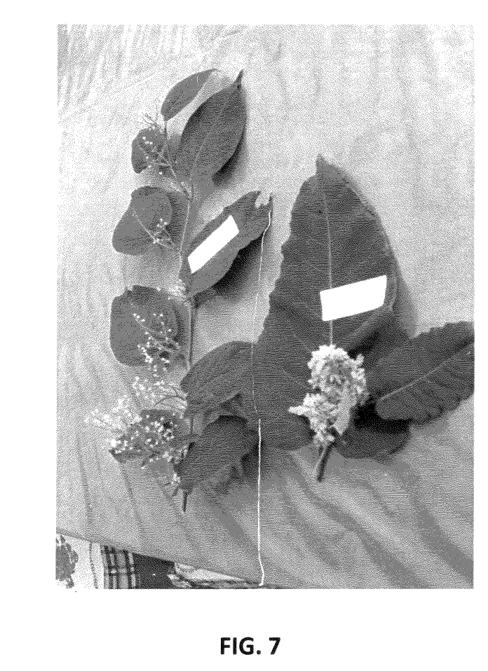

FIGS. 8A, 8B and 8C are respective photographs showing a comparison of two leaves of ‘Igniscum,’ two leaves of ‘Sachalinense’ and two leaves of ‘candy’; and

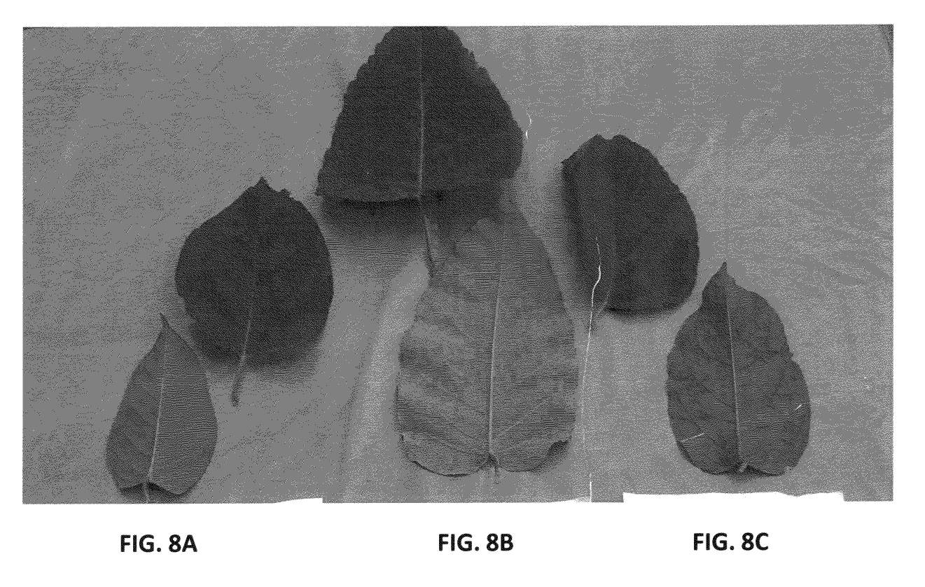

FIG. 9 is a photograph showing an enlarged view of ‘Igniscum’ leaves.

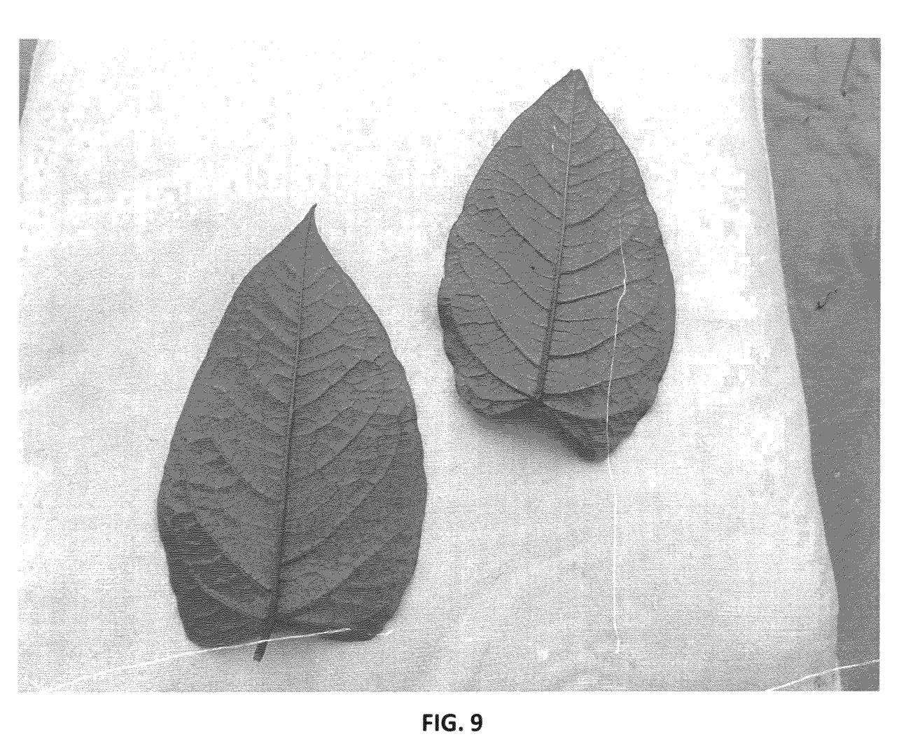

### DETAILED DESCRIPTION OF THE PHOTOGRAPHS

The following detailed description of the ‘igniscum’ cultivar is based on observations of the plant and color references are made to The Royal Horticultural Society Colour Chart, except where general terms of ordinary dictionary significance are used.

Height of the plant: 3-4 m

Site: Bright, sunny site, light to heavy soils.

Habit: No tillers, sends out runners easily, rootstock reaches down to a depth of 2 m.

Number of stems: 30-100 stems per plant.

Constitution of stem: Hollow with internodal segments, high fibre content, lignification in late summer/autumn.

Color of stem: Olive green with a slight gray shade, wipable, with insular burgundy colored dots that stretch to form lines, ceraceous layer.

Circumference of stem:

| | |
|---|---|
| at the base | 12.50 cm |
| at a height of 1 m | 11.00 cm |
| at a height of 2 m | 6.30 cm |
| at a height of 3 m | 3.14 cm |
| at the top | 1.00 cm |

Diameter of the stem

| | | |
|---|---|---|
| at the base | 3.8-4.00 | cm |
| at a height of 1 m | 3.5 | cm |
| at a height of 2 m | 2.0 | cm |
| at a height of 3 m | 1.0 | cm |
| at the top | 0.3 | cm |

Distance between nodes

Beginning with the stem, the distance between nodes starts with a distance of 16.5 cm and, with further narrowing of the stem, ends at 3 cm at the top. The stem is separated into parts by a closed lignified wall at each node.

Number of nodes

There are approximately 20-22 nodes.

Lateral shoots:

The lateral shoots are produced on the upper third of the stem. They have a length of approximately 0.45 cm -1.50 m. There are approximately 18-23 lateral shoots on each stem.

Leaf size:

Leaf size varies between a breadth of 13 cm and a length of 21 cm and a breadth of 17 cm and a length of 22 cm.

Leaf form: Elliptical, with twisted top, straight line at the lower margin, leaf color is moss green with a velvety upper surface texture at leaf maturity. Lower surface coriaceous, the leaves are very firm and with little moisture. The margins of the leaves are smooth, the lateral nerves connect at the side of the leaf in a roof-tile structure. The leaf stalks are burgundy even at maturity.

Blossoms: Creamy white.

Fresh biomass: Approximately 100 stems were harvested in an experimental harvest in the presence of an independent reviewer. The yield of fresh biomass amounted to approximately 80 kg per plant. The date of the experimental harvest was Aug. 10, 2006.

Dry biomass: Dry biomass (including leaves) is estimated to amount to approximately 20% of the calculated fresh biomass (harvest in late summer/beginning of autumn=mid-September-end of October). No leaves are left when harvesting in January. This means that the crop yield is reduced to the already dried stems.

Cold hardiness: Reynoutria biomass which has grown early in spring is generally prone to late frost damage. However, later plant growth makes up for the loss. It has been noticed on the experimental field over a longer period of time that the species ‘Igniscum’ survived these annually recurring late frosts without noticeable frost damage.

Seed: The plant produces a very small amount of seed. Long-term observation showed that the seed does not germinate. It is, apparently, sterile.

Blooming time: Depending on the weather, blooming may already start in July. Usually, blooming time is between August and November. Hybrid varieties of Reynoutria are excellent bee plants because of their late blooming time.

The following information is taken from the Plant Primer Instructions for Planting and Cultivation of the Hybrid Variety of Reynoutria ‘IGNISCUM.’

‘Igniscum Hybride’ is an energy crop belonging to the field of renewable primary products with unique properties, which should be exploited for the purpose of sustainable energy supply.

### PREPARATION OF THE FIELD

a) Site Requirements

Reynoutria hybrid forms require a soil quality of at least 30-50 soil points, however, not only on the surface but also at a depth of 0.70 m. The soil conditions should be moist, deep, and without water-logging.

It is strongly recommended not to use soils in the proximity of rivers or waters (due to the danger of flooding, wash-out and soil erosion). It is important that no parts of the plant or its roots be in contact with watercourses.

It is advantageous for the cultivation of ‘Igniscum’ to use mild clay soils with humid up to very humid conditions, because sufficient supply of water is vital for successful cultivation and high crop yield. Annual precipitation of at least 440 mm is a precondition in this context. The fast-growing ‘Igniscum Hybr’ prefers nutritious, well-aired soils that warm up quickly in spring.

‘Igniscum’ plantations should be cultivated in sites having a soil depth of at least 70 cm, a soil value number of at least 30 soil points, and the soil should have a pH value of between 5.5 and 6.5. It is not recommended to cultivate fast-growing ‘Igniscum’ if the soil value number is below 30 soil points, unless one accepts a lower production of biomass.

For wind-exposed sites, it is recommended to grow a shelterbelt with, for example, Miscanthus sin. Giganteus or other species.

b) Soil Preparation

The area to be cultivated should have been treated with alfalfa or identical nitrogen collecting plants in the previous year. Before planting, it is recommended to provide a winter furrow, and, before planting, to harrow the dry soil (harrow or power harrow) in spring. Before planting, the soil should be aerated up to a depth of 10-25 cm.

Directly before planting, green manure is sprayed with herbicides such as ‘Basta’.

A soil sample to test for N is taken directly before planting in order to allow additional manuring before the start of the growing season.

The soil must be analyzed annually for N, P, K (N on an annual basis, P and K every 2-3 years) before the start of the growing season.

Possible forms of N doses:
Deposit of N fertilizer
2-3 doses of liquid manure
Dung
Mineral fertilizer (e.g., KAS)

Doses of manure (dry matter harvest in the spring without leaves)
N—30-50 kg/ha
   1. P₂O₅ 30-50 kg/ha
   2. K₂O 50-100 kg/ha
Liquid manure
Dung
Mineral fertilizer

Doses of manure:

Reserve fertilizing

| | |
|---|---|
| In the year of planting: | 16 g N/m2 = 160 kg N/ha |
| under extensive production conditions: | 24 g N/m2 = 240 kg N/ha |
| under intensive production conditions: | 30 g N/m2 = 300 kg N/ha |

The required doses of manure can be applied very well by using compost/sewage sludge (of guaranteed quality). It is noted that liquid manure and sewage sludge promote the development of new shoots. Care must be taken with doses above the leaf carpet. It is recommended instead to work manure into the soil.

In the case of acid soil, the soil should be enhanced with a dose of lime.

### DELIVERY OF THE SEEDLINGS

The ‘Igniscum’ seedlings are delivered by plant transport with appropriately equipped transporters. A truck is equipped with a lifting platform. The plants are packed in boxes for shipping on CC roll containers. Before loading is started in the manufacturing enterprise, quality control by an authorized company is to be carried out, so that shipping is carried out with certified documents.

If it is not possible to plant the seedlings within 24 hours, it must be guaranteed that the plant material receives adequate care.

This includes:

Unloading from the roll containers to a bright site (no storing in dark barns, halls, or cold storage houses).

Avoid draft and direct sunlight.

Supply the plant with water (the plants should be moistened, but not dripping wet, which would result in the standing of the seedlings in water, thus causing the danger of root rot).

The seedlings should not be yanked out of their containers by force because that would damage the roots.

### PLANTING OF THE SEEDLINGS

In the year of planting, a seedling culture is not able to assert itself against the accompanying vegetation, which is often abundant in fields and disused areas, due to their fresh and still unlignified shoots. A precondition for optimum cultivation is therefore the preparation of a good nursery bed.

a) Planting time

Planting time always depends on the weather and the climatic conditions of the surface to be planted. In general, with experience, the plant can be cultivated during the entire growing season.

From long-term experience, the planting of perennial plants after the Ice Saints (May 11-15) is the most favorable point in time.

A further option is to plant already grown seedlings in the spring or in the autumn. The seedlings are planted by a vegetable planter into the prepared, slightly moist, pre-manured, crumbly, stone-free and clot-free cropping soil.

The seedlings must be put into the earth at a depth of at least a root ball, preferably of a root ball plus 2-3 cm.

The space between seedlings is to be adapted to the mechanical equipment needed for weed control.

After planting, in order to ensure rooting of the seedlings, it is important to keep the planted area moist; i.e.; in case of draught this area must be irrigated until the seedlings have rooted.

It is recommended to slightly earth up the plants.

In the year of planting, it is important to provide for sufficient water. Sufficient precipitation is at least 440 mm evenly distributed over the year.

b) Plant spacing

7500 plants are planted per hectare of cropping soil. According to experience, the space between the rim of the field and the plants should be +5m.

The space between rows is 0.75 m.

The space between plants in a row is 0.80 m.

It is recommended to orientate the rows towards the south in order to benefit from the most effective solar radiation.

In order to avoid a possible touching of the grown plants by a plough or grubber from a neighboring field and a following return to the wild of the plant, it is recommendable to build a root barrier around the planted area.

| | |
|---|---|
| x . . . 0.80 . . . x . . . 0.80 . . . x . . . 0.80 | space between plants, |
| | staggered row |
| | 0.75 m space between rows |
| . . . x . . . 0.80 . . . x . . . 0.80 . . . x . . . | space between plants, |
| | staggered row |
| | 2.50 m, space between lanes |
| x . . . 0.80 . . . x . . . 0.80 . . . x . . . 0.80 | space between plants |
| | 0.75 m space between rows |
| . . . x . . . 0.80 . . . x . . . 0.80 . . . x . . . | space between plants |

c) Planters

For very small plots, it is recommended to plant manually applying a garden line and a trowel.

In large-scale cultivation, forest planting machines or vegetable planters are applied, which can be adjusted to the required space between plants.

The space between plants and rows is regulated according to the machinery used for care and harvest (track width, trafficability of the field, double row design).

### CARE OF THE SEEDLINGS

Plant protection

It is decisive for a healthy development of the plants to eliminate and suppress any competing plants in the year of rooting.

Since Reynoutria is regarded a weak competitor in the year of rooting, it is required to avoid and fight weeds in the first year.

It is not recommended to plant directly after ploughing up grassland.

In the year of rooting and, if necessary, in the first year of crop, it is necessary to apply weed control measures in order to guarantee undisturbed development. In doing so it is important to prefer mechanical weed control measures to chemical ones. Precautionary measures include the selection of sites and care of the crop.
1. Preparation (cse soil preparation)
2. Mechanical weed control measures applying a currycomb, hoe, tined weeder, etc. In the first year, hoe 2-3 times. (When hoeing, be careful that no root parts are displaced by accident).
3. Option of undersown crops (lawn grass or clover)
4. Mulch plants in a bed of straw in the rows (as is done in strawberry cultures); example: ‘Amazone’ mulcher

Since no pest animals are known to this day, it is not necessary to treat the plantation with pesticides.

However, it is recommended to watch out for infestation by pests in the first years because of the monoculture.

### LATE FROSTS

From experience, a drop in the crop caused by late frosts (May-June) does not influence the crop when harvesting in the autumn. However, it is possible that strong late frosts (May-June) may cause a reduction of the biomass yield (in the case of several harvests per year).

### BIOMASS HARVESTING

1. According to biomass development, it is possible to harvest several times in the period of growth.
2. In the first year of cultivation (second year after rooting) it is recommended to only hoe the plants, in order to motivate the plant material to produce ‘eyes.’ The amount of biomass is not significant in the first year. Harvesting can be started with the 2^{nd} year (3^{rd} year after planting).
3. Fresh biomass harvesting begins at the end of June/beginning of July in a rhythm of 4-6 weeks, if it is intended not to harvest any dry matter.
4. If it is intended to harvest dry matter, fresh biomass should exclusively be harvested only once. Dry matter should exclusively be harvested in the winter months (January/end of February) with the start of the frost period in order to avoid damaging the young shoots, which under mild weather conditions grow very early below the surface.
Harvesting is recommended when the soil is frozen in order to protect the root balls and avoid soil compaction.

Harvesting is carried out as closely as possible above the soil surface (root balls must not be damaged).

### HARVESTING MACHINERY

To this date, biomass harvesting has been carried out in experiments using row-independent corn choppers.

It is also possible to use a harvesting machine or a chopper as is used in the forest and lumber industry for harvesting biomass for the production of energy.

The lumber industry already has fully automated wood chip harvesters available.

### ECOLOGICAL CONSEQUENCES

The cultivation of ‘Igniscum Hybr’ in fields is characterized by a conservative application of fertilizers and pesticides. This crop cultivation sustainably extends the possibilities of ecological agriculture with a lasting effect.

1. A new and distinct variety of Fallopia plant named ‘Igniscum,’ substantially as shown and described herein.
